(19) United States
(12) Patent Application Publication
(10) Pub. No.: US 2003/0199839 A1
(43) Pub. Date: Oct. 23, 2003

(54) Eye drops guide

(76) Inventors: Joseph Kedmi, Jerusalem (IL); Benlumin Sharon, Rehovol (IL)

Correspondence Address: KATTEN MUCHIN ZAVIS ROSENMAN, 575 MADISON AVENUE, NEW YORK, NY, 10022-2585, US

(21) Appl. No.: 10/414,948

(22) Filed: Apr. 16, 2003

Related U.S. Application Data
(60) Provisional application No. 60/373,347, filed on Apr. 18, 2002.

(51) Int. Cl. A61M 35/00

(52) U.S. Cl. 604/295

(57) ABSTRACT

A device to be used with an existing eye-drop dispenser in order to assist the user in the self-administration of eye medication in the form of eye drops. The device comprises of a concave mirror, attached to the eye-drop container in a position where the mirror is at an angle approximately 35 degrees from the vertical axis of the container. When the reflected image of the eye is in focus, the eye-drops dispensed from the container reaches the correct area of the eye while an enlarged image of the eye is clearly visible during the process.

This application claims benefit under 35 U.S.C. §119(e) of earlier filed Provisional Application No. 60/373,347, filed Apr. 18, 2002, which was filed by at least one inventor common to the present application and which is hereby incorporated by reference.

### BACKGROUND OF THE INVENTION

Generally, eye-drops are inserted into an eye using a dispenser that is positioned above the eye while the eye is positioned horizontally, i.e. facing upwards. It is normally difficult for a user to direct the eye-drops to his/her own eye without help from other. The need arises for a device that will assist the user in inserting eye-drops into his/her own eyes. One method to achieve this is to attach a mirror to the eye-drop dispenser in a way such that the user can see his/her eye in order to direct the eye-drops to it.

Several patents describe this method of assisting in the self-administration of eye-drops. Such a patent is European Patent no. 197,344, which describes a device comprising of a large mirror with an angled extension. The extension has a round protruding border designed to hold the bottom of an eye-drop container of a specific size. The large size of this device and its incompatibility with eye-drop containers of varying sizes and shapes may prove disadvantageous.

A similar patent, U.S. Pat. No. 4,344,430, describes a device comprising of a relatively smaller mirror and an extension to hold the bottom of the eye-drop container. The means of clamping the eye-drop container in place is mainly dependent on adhesive, which may not hold the eye-drop dispenser in place reliably. Furthermore, it would be difficult or impossible to use this device many times with different eye medication containers.

Finally, U.S. Pat. No. 3,779,245 describes a much more compact and convenient solution. However, here, a small mirror is permanently attached to a specific container of eye medication, requiring that the eye-drop container and assisting apparatus be distributed together as one unit. The user is not able to remove or reattach the device to other eye-drop dispensers.

Each of the patents described above has a further disadvantage; the mirror is placed at the far end of the container (i.e. further away from the eye of the user while s/he is inserting the eye-drops). Therefore, the need for an eye-drop guide apparatus that is compact, convenient, versatile and easy to use is still present. The present invention aims to achieve these goals, as well as to add further features, such as enlargement of the reflected image of the eye.

### SUMMARY

The present invention discloses an apparatus for assisting in self-administration of eye drops, wherein the apparatus can be attached to the rim of eye drop dispenser, said apparatus comprised of a concave mirror and a grip designed to grasp the dispenser rim, wherein the concave mirror is angled relative to the axis of the eye drop dispenser.

### BRIEF DESCRIPTION OF THE DRAWINGS

A more complete understanding of the inventions may be obtained by reading the following description of the specific illustrative embodiment of these invention in conjunction with the appended drawing in which

FIG. 1 is an illustration of eye-drop guide while attached to an eye-drop dispenser in accordance with a preferred embodiment of the present invention;

FIG. 2A is an enlarged perspective view of the eye-drop guide in accordance with a preferred embodiment of the present invention;

FIG. 2B is an enlarged top view of the eye-drop guide in accordance with a preferred embodiment of the present invention;

FIG. 3 is an illustration of eye-drop guide while attached to an eye-drop dispenser in accordance with the fourth alternative embodiment of the present invention;

FIG. 4A is an enlarged perspective view of the eye-drop guide in accordance with the fourth alternative embodiment of the present invention;

FIG. 4B is an enlarged top view of the eye-drop guide in accordance with the fourth alternative embodiment of the present invention;

FIG. 5 is an illustration of the eye-drop guide while attached to an eye-drop dispenser in accordance with the fifth alternative embodiment of the present invention;

FIG. 6 is an illustration of the use of the eye-drop guide in any of the preferred embodiments in accordance with the present invention;

FIG. 7 is an enlarged perspective view of the eye-drop guide in accordance with a first alternative embodiment of the present invention;

FIG. 8 is an enlarged perspective view of the eye-drop guide in accordance with a second alternative embodiment of the present invention; and

FIGS. 9A, 9B are enlarged perspective views of the eye-drop guide in accordance with a third alternative embodiment of the present invention.

### DETAILED DESCRIPTION OF THE PREFERRED EMBODIMENTS

The present invention provides a device for helping the user to insert eye drops into his/her own eyes by adding a strategically placed mirror to the existing eye drop dispenser. This mirror assists the user in positioning the eye drop dispenser directly above the portion of the eye in which s/he wishes to insert the eye drops without the help or direction of others, enlarging the reflection of the eye in the process.

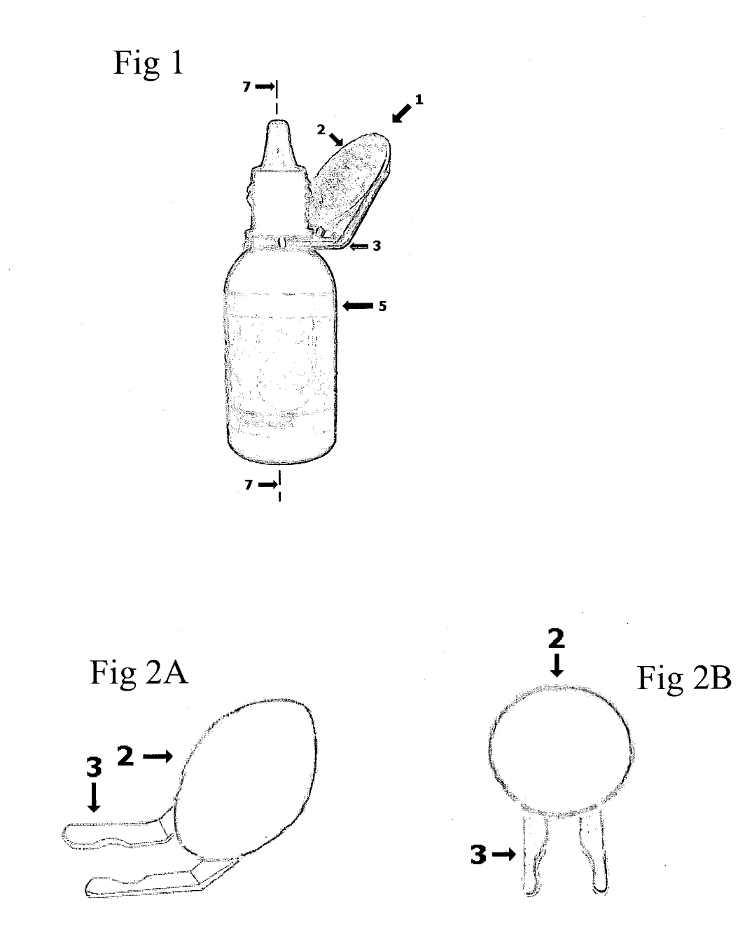

Reference is made to FIG. 1, which illustrates an eye-drop guide 1 constructed and operative in accordance with the preferred embodiment of the present invention. According to this embodiment, the eye-drop guide must be attached to the upper rim of an existing eye-drop dispenser—just below the nozzle—for use, and may be distributed separately from the eye-drop medication containers and used and reused any number of times with as many different eye-drop containers as the user wishes. The eye-drop guide 1 comprises of a concave mirror 2, and a flexible grip 3 for connecting the mirror 2 to an eye-drop dispenser 5. According to the preferred embodiment of the present invention the mirror is spherically concave with a radius of curvature of approximately 65 mm. The grip 3 positions the mirror 2 at an angle with the vertical axis 7 of the container 5. According to the preferred embodiment of the present invention the angle may be approximately 35 degrees. The grip 3 is made of two semicircular prongs and grips the container 5 at its rim using spring force, thus making it possible for the user to easily detach the eye-drop guide from one container and attach it to another.

FIGS. 2A and 2B illustrate two views of the eye-drop guide constructed in accordance with the preferred embodiment of the present invention. The eye-drop guide comprises of a concave mirror 2 and a flexible grip 3.

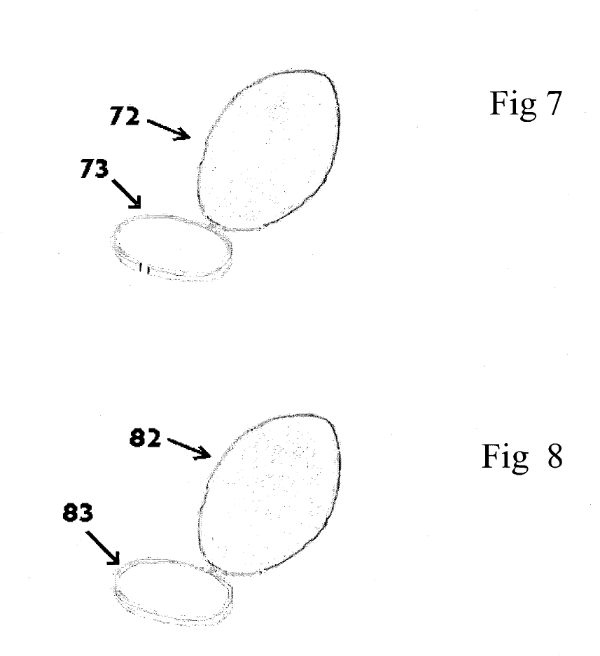

According to a first and second alternative embodiments of the present invention, the grip may take the form of either an open flexible ring 73 (see FIG. 7), which may easily be attached to the eye drop container, or a closed flexible ring 83 (See FIG. 8).

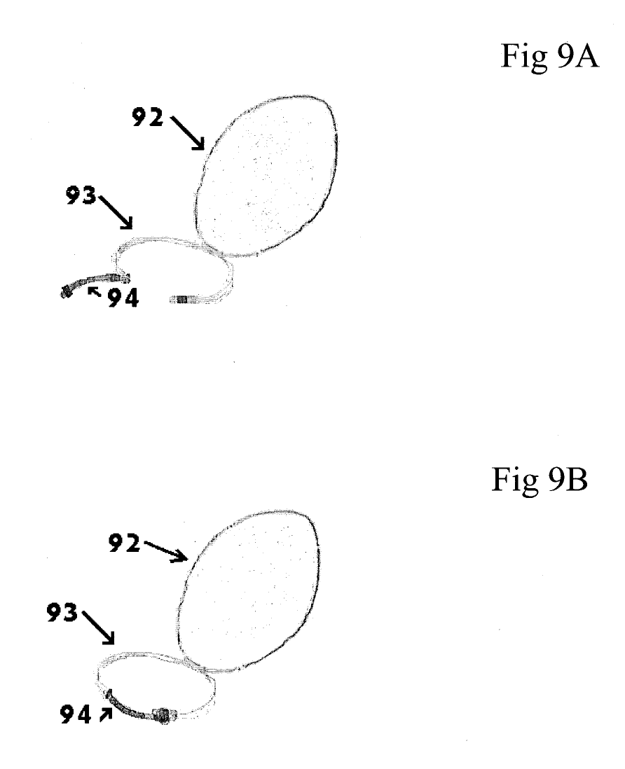

According to a third alternative embodiment of the present invention the grip 93 may take the form of an open ring having a fastener clip for closing the ring (see FIGS. 9A and 9B).

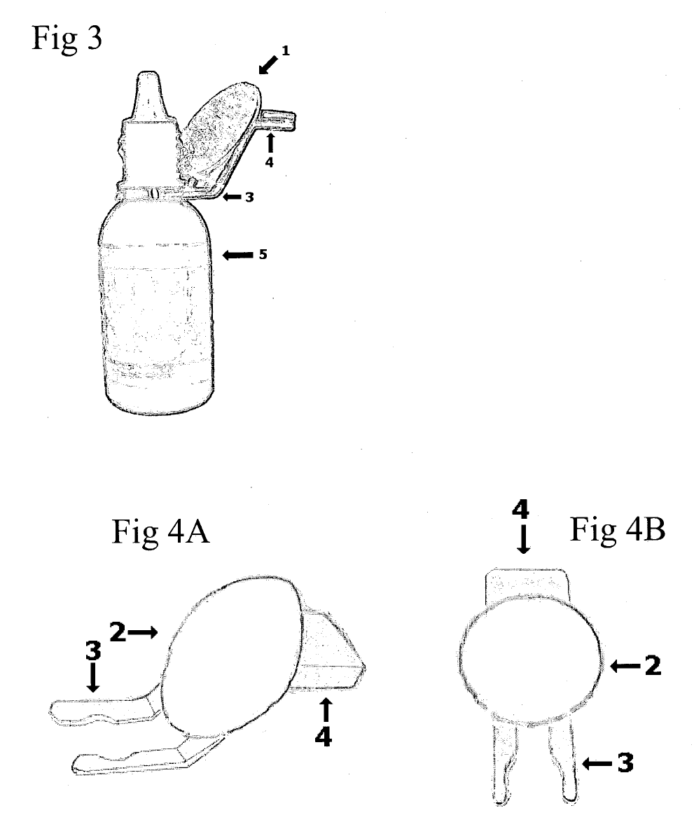

Reference is now made to FIG. 3, which illustrates an eye-drop guide 1 constructed and operative in accordance with a fourth alternative embodiment of the present invention. This embodiment is similar to the first embodiment in that it requires attachment to the upper rim of an existing eye-drop dispenser for use, and comprises of the same components as the first embodiment, with the addition of an extension 4 to the grip 3. The extension 4 serves as a handle to allow easy attachment and detachment of the eye-drop guide 1 to and from the eye-drop dispenser 5.

FIGS. 4A and 4B illustrate two views of the eye-drop guide constructed in accordance with the fourth alternative embodiment of the present invention. The eye-drop guide comprises a concave mirror 2, a flexible grip 3, and an extension 4.

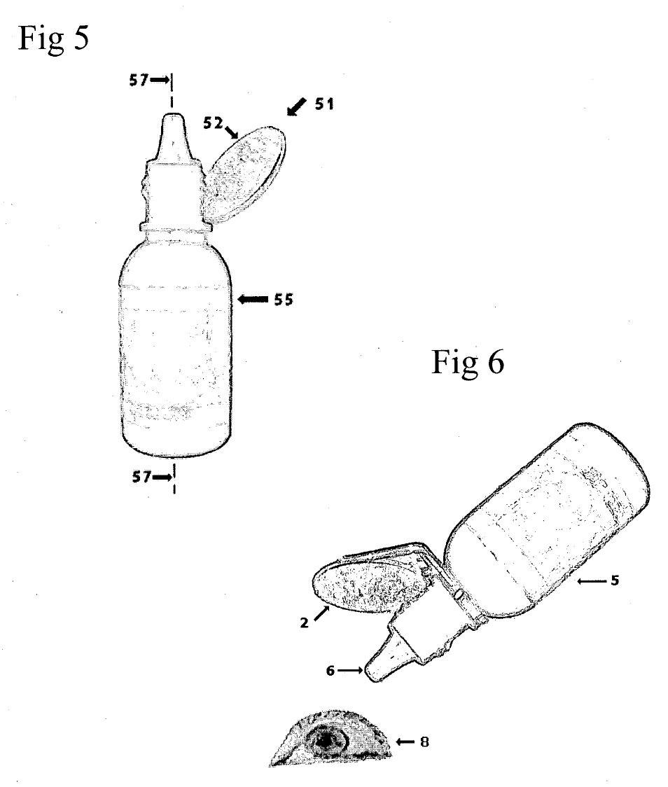

Reference is now made to FIG. 5, which illustrates an eye-drop guide 51 constructed and operative in accordance with the fifth alternative embodiment of the present invention. According to this embodiment, the eye-drop guide is permanently attached to the upper rim of an eye-drop dispenser and may not be removed or reattached, and may be distributed with containers of eye-drop medication. The eye-drop guide 51 comprises of a concave mirror 52, similar to that in previous embodiments, that is permanently attached to the container 55 at an angle of approximately 35 degrees from the vertical axis 57 of the eye-drop dispenser 55.

Reference is now made to FIG. 6, which illustrates the use of the eye-drop guide. When the user brings the reflected image of the eye 8 to focus in the mirror 2, the tip 6 of the eye-drop container 5 is at a position that ensures an accurate insertion of the eye drops to the eye. When the image of the eye 8 is in focus, the tip 6 of the eye-drop container 5 from which drops are dispensed is at a safe distance from the eye 8. In addition, the concave mirror 2 provides an enlarged view of the eye 8 and of the container's nozzle 6, so that the user can insert the drops accurately into any part of the eye. When the concave mirror 2 is positioned in a plane that is approximately parallel to the plane of the eye 8, the eye-drop container 5 is positioned at approximately 35 degrees to the plane of the eye so that the drops can be easily dispensed from the container.

While the above description contains many details, these should not be construed as limitations on the scope of the invention, but rather as exemplifications of the preferred embodiments. Those skilled in the art will envision other possible variations that are within its scope. Accordingly, the scope of the invention should be determined not by the embodiment illustrated, but by the appended claims and their legal equivalents. This description describes the invention in terms of embodiments foreseen by the inventors for which an enabling description was available, notwithstanding that insubstantial modifications of the invention, not presently foreseen, may nonetheless represent equivalents thereto.

What is claimed is:

1. An apparatus for assisting in self-administration of eye drops, wherein the apparatus can be attached to the rim of eye drop dispenser, said apparatus comprised of:
a. a concave mirror;
b. a grip designed to grasp the dispenser rim;
wherein the concave mirror is angled relative to the axis of the eye drop dispenser.

2. The apparatus of claim 1 wherein the mirror is spherically concaved.

3. The apparatus of claim 1 wherein the grip is flexible and has two prongs wherein said prongs are designed to grasp the dispenser rim.

4. The apparatus of claim 1 wherein the grip is a flexible open ring designed to grasp the dispenser rim.

5. The apparatus of claim 1 wherein the grip is a flexible closed ring designed to grasp the dispenser rim.

6. The apparatus of claim 1 wherein the grip is an open ring having attachment means for closing the ring said attachment means designed to grasp the dispenser rim.

7. The apparatus of claim 1 wherein the angle between the concave mirror and the axis of the eye drop dispenser is 35 degrees.

8. The apparatus of claim 1 wherein the grip further includes an extension wherein said extension serves as a handle for the purpose of attaching or detaching the apparatus.

9. An apparatus for assisting in self-administration of eye drops including a concave mirror wherein the concave mirror is permanently attached at the dispenser rim and angled relative to the axis of the eye drop dispenser.

10. The apparatus of claim 9 wherein the angle between the concave mirror and the axis of the eye drop dispenser is 35 degrees.

11. The apparatus of claim 9 wherein the mirror is spherically concaved.
